(19) United States
(12) Patent Application Publication
(10) Pub. No.: US 2025/0127458 A1
(43) Pub. Date: Apr. 24, 2025

(54) MEASURING ARRANGEMENT AND METHOD FOR MEASURING MOVEMENTS OF A PATIENT POSITIONED RECUMBENTLY ON A PATIENT TABLE, AND MAGNETIC RESONANCE DEVICE

(71) Applicant: Siemens Healthineers AG, Forchheim (DE)

(72) Inventor: Stefan Popescu, Erlangen (DE)

(21) Appl. No.: 18/920,961

(22) Filed: Oct. 20, 2024

(30) Foreign Application Priority Data
Oct. 20, 2023 (DE) 10 2023 210 366.4

(51) Int. Cl. A61B 5/00; A61B 5/055

(52) U.S. Cl. CPC A61B 5/721; A61B 5/055; A61B 5/704; A61B 2562/0266

(57) ABSTRACT

A measuring arrangement for measuring movements of a patient positioned recumbently on a patient table includes the patient table and a securing strap for locally securing the patient on the patient table. The securing strap is flexible and has at least one first fastener that co-operates with a second fastener of the patient table for fastening the securing strap. The measuring arrangement includes a strain sensor provided as part of the securing strap and/or motion-coupled thereto, and a control device for determining motion information describing the movements of the patient from sensor data of the strain sensor.

This application claims the benefit of German Patent Application No. DE 10 2023 210 366.4, filed on Oct. 20, 2023, which is hereby incorporated by reference in its entirety.

### BACKGROUND

The present embodiments relate to measuring movements of a patient positioned recumbently on a patient table.

For examinations and treatments using medical imaging devices, for example, patients are often placed on patient tables. This applies, for example, to examination and treatment methods that are sensitive to patient movements. This includes imaging procedures with acquisition modes that take a significant amount of time. Computed tomography, positron emission tomography (PET), and magnetic resonance imaging (MRI) are typical examples of imaging modalities in which motion that may occur during the acquisition time may result in quality impairments (e.g., artifacts) in the images.

For example, patient motion that is undetected or uncompensated, especially computationally, may result in severe image artifacts that may render the entire image dataset useless. Low-quality image datasets also hinder consistent clinical assessment and diagnosis as well as the monitoring of treatment procedures. For example, magnetic resonance imaging is susceptible to patient motion due to the long duration of the magnetic resonance imaging sequences (MRI sequences).

Patient motion includes both unconscious movement, such as twitching or muscle spasms, and cyclical movement, such as respiration and heartbeat, as well as intentional movements of the patient. Consequently, a reliable method for detecting and quantifying patient motion would be useful in order to re-acquire at least some of the image data that is unusable due to motion and/or to trigger image data acquisition for time intervals with little or no motion and/or to implement various algorithms for motion correction and/or motion compensation.

Solutions for detecting patient movement have already been proposed in the prior art. A commonly applied example involves mechanical transducers, such as respiratory belts and the like, that, however, like EKG devices that electrically measure the heartbeat, may cause discomfort to the patient and/or overload the workflow during image data acquisition. For example, additional staff time and expertise are required to attach the sensors to the patient and put the sensors into operation.

In general areas of application, it has also been proposed that the patient be continuously monitored remotely by sensors (e.g., by at least one video camera that captures images of the patient). For the analysis, landmarks on or around the patient may be tracked to infer patient movement. However, problems arise if the direct line of sight between the sensor and the patient is obstructed (e.g., by a blanket covering the patient or by devices placed on or around the patient).

In the field of magnetic resonance imaging, for example, other approaches have already been identified for detecting and ideally quantifying patient movements.

An initial example of this is the pilot tone method. It has been proposed, for example, to incorporate a special pilot tone transmitter in the patient table (e.g., under or within the positioning surface for the patient), which emits a pilot tone signal that is close to the Larmor frequency. The pilot tone signal is received and evaluated by receive coils (e.g., a local coil array disposed on the patient). However, such a solution has been found to be sensitive to external interference, such as that caused during magnetic resonance imaging by the coldhead of the cooling device, the gradient coil cables, and the like. Significant modifications are to be made to the control software of the magnetic resonance device in order to allow continuous operation of the pilot tone receive chain. This also applies, for example, outside the readout periods. In addition, the procedure is heavily dependent on the skillful placement of the receive coils by the operator. Another extremely relevant problem is the inability of the pilot tone method to detect patient movements that take place away from the active receive coil but may nevertheless produce field fluctuations of the main magnetic field in the field of view, especially at high main magnetic field strengths. For example, during a high-field head examination (e.g., at 3 to 7 tesla), effects due to respiratory motion that cannot be measured due to the lack of a receive may occur. In addition, the frequency of the pilot tone signal is to be reselected for each field strength. In general, the approach is not compatible with multicore magnetic resonance imaging.

In another approach, it has been proposed, for magnetic resonance imaging, to incorporate magnetic field sensors (e.g., Hall sensors) in the local coil array. This type of patient movement detection is based on the ability of the magnetic field sensor to detect its own movement (e.g., induced by movement of the local coil array) in the strong homogeneous magnetic field within the field of view (e.g., homogeneity volume). One problem here, however, is that if the magnetic field sensor moves in parallel such that its angle relative to the direction of the main magnetic field remains constant, no field variation occurs and therefore even significant patient motion cannot be detected.

Both the approach using magnetic field sensors and the pilot tone approach share the common requirement of using new types of local coil arrays that are to be specially distributed across existing magnetic resonance devices. These local coil arrays include dedicated, built-in electronic units. Users would not be able to use local coil arrays with new magnetic resonance devices or use local coil arrays from a manufacturer that does not also provide the magnetic resonance device. Consequently, the costs increase, and the implementation of these technologies is restricted.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved, precise way of detecting movements of a patient positioned on a patient table that may be implemented in a simplified manner, in particular, for magnetic resonance imaging is provided.

According to the present embodiments, a measuring arrangement of the type mentioned in the introduction also has at least one strain sensor provided as part of a securing strap and/or motion-coupled thereto at least in a fastened state via a fastening means (e.g., a fastener), and a control device for determining motion information describing the movements of the patient from sensor data of the strain sensor.

The present embodiments are therefore based on the idea that auxiliary devices to be disposed particularly on and/or around the patient, which may be used, for example, during examination and/or treatment, and/or the patient themselves are directly secured at least locally to the patient table by at least one securing strap. Patient movements thus act directly or at least indirectly (e.g., via the auxiliary devices) on the securing strap, so that tensile forces or a tensile force variation is generated there, which describes the patient movement. Such tensile forces may be detected with a suitably positioned strain sensor (e.g., at least one strain sensor for each securing strap). The resulting measurement signal describing the forces acting on the securing strap (e.g., the sensor data) describes the patient movement causing the measured forces. The sensor data may therefore be evaluated in a control device of the measuring arrangement to produce motion information that describes the movement of the patient.

As will be explained in more detail below, the motion information may be configured differently depending on the application and/or may also be focused on different types of movement. This provides that in a simple embodiment, for example, the motion information may simply indicate whether a patient movement is present that exceeds a particular motion intensity threshold. This may also relate to different movement types. However, it is also possible and preferred if the motion information quantifies recorded patient movements of at least one movement type. For example, the fixation strap may describe, possibly even broken down by movement type, how patient movements vary over time. In general, the control device for determining the motion information may also be configured to describe the location and/or the intensity and/or the type of patient movement.

The motion information may be used in various ways in the course of examining and/or treating a patient (e.g., to trigger acquisition procedures and/or treatment procedures, to identify acquisition data corrupted by motion, and/or to correct and/or compensate for motion in acquired data). For example, the present embodiments are carried out in the context of medical imaging, where the control device may be the control device of a corresponding imaging device. Its use in magnetic resonance imaging, which is particularly susceptible to patient motion, is advantageous. Here, as with other imaging modalities, it may be provided that the motion information is used by the control device to reacquire at least some of the image data that is unusable due to motion and/or to trigger image data acquisition for time intervals with little or no motion and/or to execute various motion correction and/or motion compensation algorithms that use the motion information as input data.

Within the scope of the present embodiments, movement types may include, for example, cyclical patient movements, such as heartbeat and/or respiration, which may be easily determined by appropriate evaluation functions due to their periodicity and the known frequency range in the sensor data. However, sporadic movements may also be captured (e.g., unconscious movements and/or conscious movements of the patient) as movement types.

An evaluation function of the control device that evaluates the sensor data relating to the motion information may self-evidently also use other information in addition to the sensor data as input data. For example, background information may also be taken into account, which, for example, describes the path of mechanical interactions triggered by the motion to the strain sensor. For this purpose, the control device may specifically take into account background information available in a memory device of the control device and relating to properties of the securing strap and/or of the auxiliary devices and/or of the type of fastening when determining the motion information.

As already mentioned, the measuring arrangement may be used advantageously as part of or for a magnetic resonance device in the context of magnetic resonance imaging. In magnetic resonance imaging, local coil arrays including at least one transmit and/or receive coil may be used as an auxiliary device (e.g., to detect the weak magnetic resonance signals that are induced in various tissues within the human body and emitted therefrom). Many local coil arrays known today include a plurality of receive coils that are spatially distributed in two-dimensional or three-dimensional matrix-or array-like configurations. For example, the local coil array may be sufficiently flexible to bend the local coil array over the anatomy of the patient. To reduce high-frequency noise in the received magnetic resonance signals, the receive coils are brought as close as possible to the body of the patient. In order to prevent image artifacts and due to the sensitivity profiles of the receive coils changing with the relative position to the patient anatomy, the local coil array is to remain in the original position in which the calibration of the sensitivity profiles took place, without moving or slipping.

To achieve this, it has already been proposed in the prior art to provide local coil arrays having at least one securing strap (e.g., a plurality of securing straps) to bend the local coil array and fasten the local coil array around the body of the patient while at the same time constraining the local coil array so that the local coil array remains in place throughout the entire examination. The at least one securing strap, with at least one pair of oppositely disposed securing straps usually being provided, may be fastened to the local coil array via a fastening device (e.g., a corresponding mount in a support of the local coil array) and may be adjustable in its free length. At the opposite end, the at least one securing strap has a first fastening means (e.g., a first fastener such as an insertable connector) that may be fastened to a second fastening means (e.g., a second fastener) on the patient table (e.g., an insertion receptacle). The adjustability of the free length of the at least one securing strap used for fastening allows the user to adjust the mechanical tension in the securing strap, such that patient discomfort is avoided and any change in position of the local coil array or other auxiliary device is prevented.

With regard to these and other generally known securing straps, the present embodiments utilize the practical observation that any type of patient movement is automatically translated into a variation of the tensile forces received via the securing straps.

For example, as just explained in more detail, in magnetic resonance imaging, if a local coil array is fastened with four securing straps at four fastening points on the patient table (e.g., two points on the left side of the patient, such as by an insertion channel, and two points on the right side of the patient, such as against another insertion channel), the securing straps are subject to varying tensile forces. The varying tensile forces (e.g., in the case of flexible but at least essentially non-extensible securing straps) are also transmitted to the second fastening means (e.g., second fastener) of the patient table, where the varying tensile forces are translated, for example, into a variable tensile force on the second fastener (e.g., the insertion channel).

In other words and in general terms, any type of unconscious or conscious movement of the patient, such as breathing, heartbeats, and/or muscle spasms, produces mechanical vibrations of low amplitude along the securing straps, and in the case of non-extensible (e.g., inelastic, securing straps), all the way to the second fastener on the patient table. This becomes especially noticeable in the case of significant patient movement, such as movement caused by coughing, pain, or panic.

Depending on the examination, it may be necessary to use a plurality of securing straps along the length of the patient (e.g., in order to attach a plurality of auxiliary devices such as local coil arrays). For whole-body magnetic resonance imaging, for example, local coil arrays and corresponding securing straps are distributed over a plurality of locations along the human body so that patient motion at these different locations along at least one or, for example, both sides of the patient may be captured by strain sensors. In such cases, unlike methods known from the prior art that are based, for example, on the reception of signals by receive coils, patient movements outside the current field of view may also be detected (e.g., at a distance from an active receive coil and even far removed from the current homogeneity volume relative to the patient table and the patient).

Generally speaking, it is provided within the scope of the present embodiments to use strain sensors to measure patient motion captured by at least one securing strap (e.g., by a plurality of securing straps). A strain sensor is a sensor that measures tensile forces resulting in deformation of a measuring element, such as tension and/or bending. In the context of the present embodiments, a strain sensor may, for example, include a sensor element that is subjected, directly or indirectly (e.g., by being attached to the measuring element), to the forces to be measured, and is thus deformed. The sensor element itself is often made of a material that is soft and extensible (e.g., elastic). The strain sensor may also include a control unit to derive the sensor data from signals from the sensor element and/or from properties of the sensor element. A variety of different types of strain sensors that may also be used in the context of the present embodiments have already been proposed in the prior art. Known types of strain sensors include, for example, rheostatic strain sensors that include an electrical conductor for which the electrical resistance changes when the sensor element is deformed. Capacitive-type strain sensors are also known in which the sensor element includes a distributed capacitor having a capacitance that varies with deformation. A third type of strain sensor uses piezoelectric materials in soft, flexible or extensible formats as the sensor element, where the ability of piezoelectric material to convert between mechanical and electrical forms of energy is utilized. Finally, an option for magnetic resonance imaging applications, which will be discussed in more detail below, is a known type of strain sensor that uses optical fibers in the sensor element.

As already mentioned, it may also be provided within the scope of the present embodiments that the measuring arrangement further has an auxiliary device (e.g., a local coil array) to be positioned on the patient and secured there, to which at least two securing straps of the plurality of securing straps are fastened by the end opposite the first fastener using a fastening device.

However, in example embodiments, securing straps independent of (e.g., separate from) auxiliary devices may also be used. It may therefore be provided that at least one of the securing straps has first fasteners on both sides for opposite second fasteners of the patient table. Securing straps of this type may be used to directly secure the patient locally on the patient table, but self-evidently also to hold auxiliary devices securely in position on the patient. For this purpose, for example, a flexible securing strap with two first fasteners may be placed over the auxiliary device and/or a target anatomy and, for example, after adjusting the length of the securing strap using an appropriate adjustment device, may be fastened to opposite second fasteners of the patient table in a transverse direction perpendicular to the longitudinal direction.

For example, a flexible securing strap that is not part of an auxiliary device (e.g., a local coil array) but may be used in combination with any auxiliary device (e.g., any local coil array) to detect movements of the patient in the body region disposed mechanically close to the flexible securing strap may thus be provided. A user may adjust the length of the securing strap to adjust the mechanical tension in the securing strap so that sufficiently good patient movement detection is possible, and an auxiliary device does not move accidentally. For example, the independent securing strap may thus be used in the context of magnetic resonance imaging to secure a flexible local coil array, such as a knee coil, a shoulder coil, a wrist coil, and the like, to the corresponding body part of the patient in order to prevent the devices from moving.

However, an independent flexible securing strap of this kind may also be used with rigid local coil arrays, such as rigid head coils or rigid knee coils of the prior art. In this case, a securing strap may be applied directly to the patient (e.g., particularly in the area of the forehead or chin or around a leg, adjacent to the knee) in order to measure the movement of the head or leg.

With particular advantage, however, such securing straps may also be used to measure the movement of various parts of the body of the patient even when an auxiliary device, such as a local coil array, is not attached to that body part. This may be done simply by placing the securing strap around the body part and fastening the securing strap using the first fastener, tensioning the securing strap sufficiently as in the case of use with an auxiliary device. Such an application scenario is advantageous, for example, if heartbeats or respiratory movements are to be tracked during an examination of the head. This may be provided in the context of magnetic resonance imaging, as magnetic field fluctuations within the field of view that may be induced by such movements may then be prevented and/or corrected.

In the case of all interconnected overall structures to be fastened on both sides of the patient table (e.g., auxiliary devices with securing straps on each side and/or securing straps that may be fixed on both sides of the patient table), sensor data may be acquired via at least one strain sensor on both sides in the transverse direction (e.g., to the left and right of the patient). Since it is assumed in the context of the present embodiments that the securing straps are fundamentally under at least a certain amount of tension (e.g., as it would not be possible to obtain a measurement if the straps were sagging/loose), even if one side is untensioned during measurement on both sides, a measurable tensile force will arise on the other side.

In the context of the present embodiment, a distinction may be drawn between two basic exemplary embodiments. In a first variant, it may be provided that a sensor element of the strain sensor (e.g., a strain gage) is provided on an elastic portion of at least one securing strap of the plurality of securing straps. In this case, the securing strap is therefore at least in part extensible/elastic and may indicate deformations in the elastic part that are caused by patient movement that may be measured by the sensor element of the strain sensor. However, signals or sensor data is to be transferred from the securing strap to the control unit via a suitable interface if the control unit is also provided by the securing strap.

In this context, it may be provided that, in the case of a securing strap attached to an auxiliary device, the sensor element is connected wirelessly or wired to a control unit of the strain sensor on or in the auxiliary device (e.g., in an electronic unit of the auxiliary device). For example, if the auxiliary device is a local coil array, it may be provided that the control unit of the strain sensor is incorporated in an already present electronics unit of the local coil array. The fastening device may provide an interface (e.g., a wired interface) for communication between the sensor element and the control unit. In this context, the sensor element may be disposed adjacent to the fastening device, as this provides a short transmission distance. Such an arrangement may be less preferable, as a special design of the auxiliary device (e.g., of the local coil array) is to be provided, which limits universal applicability.

In another embodiment, however, it is also possible for at least one of the first fasteners to have an interface for connecting the sensor element to a control unit in the patient table. In this case, only the securing strap is therefore to be specially designed. If the latter is provided independently of an auxiliary device (e.g., by a securing strap with first fasteners on both sides), as described above, a special design of the auxiliary device is no longer required. In this embodiment, the sensor element may be disposed adjacent to the first fastener in order to keep the transmission distance short.

However, in a second variant, it may be provided that the varying tensile forces to be measured are conveyed to a measuring point in the patient table via the securing strap, which in this context may still be flexible but at least not significantly extensible (e.g., is inelastic). It may therefore be provided that the securing strap for transmitting the forces to be measured is at least essentially inextensible (e.g., inelastic). However, the second variant is not precluded even if the securing strap has a certain amount of extensibility or elasticity, provided that at least some of the tensile forces (e.g., small tensile forces) to be measured are transmitted to the patient table via the first fastener (e.g., first rigid fastener) and produce an effect that may be measured by the strain sensor.

In this context, the securing strap may have an adjustment device for adjusting the length of the securing strap. Such an adjustment device may be advantageous if no change in length may be brought about by extensibility or elasticity. The adjustment device may then be used to adjust the securing strap so that the securing strap lies closely against the auxiliary device and/or the patient and is thus affected by any movements of the auxiliary device and/or the patient (e.g., follows these movements and thus transmits the corresponding tensile forces via the first fastener).

The advantage of such an embodiment, in which the strain sensor may be provided in the patient table, is that an “upgrade” is possible simply by modifying the patient table (and possibly providing suitable, non-extensible, securing straps). Auxiliary devices (e.g., local coil arrays) may continue to be used as usual. Securing straps are also often already sufficiently inextensible to allow sufficient force transmission. For example, in the case of the magnetic resonance device, it may be sufficient to replace the patient table in order to implement the measurement option according to the present embodiments. Any equipment (e.g., auxiliary devices and (inelastic) securing straps) may continue to be used.

Although a patient table measurement may be provided, it would also be conceivable to provide the strain sensor as part of a fastening device for the auxiliary device (e.g., a local coil array). However, this may not be provided due to the special design of the auxiliary device that would then be required.

Consequently, an embodiment provides that at least one strain sensor of the plurality of strain sensors is provided as part of the first fastener and/or the second fastener. The basic idea is that, for example, the tensile force may be transmitted to the second fastener, where the strain sensor may be provided, due to the non-extensibility of the securing strap, without the need to provide complex data transfers from and/or to the securing strap. In other words, by transmitting the tensile forces via the combination of first fastener and second fastener, it is possible to implement the at least one strain sensor in the patient table.

This avoids any disadvantages that may arise, such as increased complexity and increased power consumption of electronic units of auxiliary devices (e.g., local coil arrays) and/or data transfers that are difficult to implement (e.g., in magnetic resonance applications). In addition, compatibility with auxiliary devices and/or securing straps from third-party manufacturers may be achieved.

In a specific development of this embodiment of the second variant, it may be provided that the first fastener has a fastening element (e.g., a latching element) that, in the fastened state, lies closely against a measuring element of the second fastener. The measuring element is configured as a sensor element of the strain sensor, or the sensor element of the strain sensor is disposed on the measuring element. In this context, the first fastener and the second fastener may be configured, for example, to establish a latching connection and/or a clip connection. It is therefore advantageous that the fastening element may first be inserted into the second fastener against a restoring force and then snap into place there (e.g., in a cavity (receiving space) serving as a receptacle) and, in creating the fastening connection, rest closely against the measuring element of the second fastener, at least secured against being pulled out. It is also this securing against being pulled out that allows the relevant force transmission for tensile forces from the securing strap via the fastening element to the measuring element, resulting in the deformation thereof (e.g., local deformation) that may be measured by the strain sensor.

The measuring element does not require a high degree of elasticity/deformability, as modern strain sensors, which will also be discussed in more detail below, and may thus measure even minor changes in the measuring element, and thus the sensor element, with a sufficient degree of reliability and accuracy.

In a specific embodiment, it may be provided that the second fastener is configured as an insertion channel for inserting the first fastener in any position covered by the insertion channel (e.g., in the longitudinal direction of the patient table). Such insertion channels, which are known in principle, have already been proposed in the prior art and allow flexible insertion of the first fastener in any desired position longitudinally along the length of the insertion channel. For example, the patient table may have at least two insertion channels, at least one of which is provided on the right-hand side of the patient table and at least one of which is provided on the left-hand side of the patient table, at least essentially over the entire length thereof. Further, shorter pairs of insertion channels located opposite each other in the transverse direction may be provided, for example, in the region of the head and/or in the region of the arms and/or in the region of the legs.

In one embodiment, the measuring element may form a limit of an insertion slot of the insertion channel and of a cavity that is configured to accommodate the fastening element and is wider than the insertion slot. For example, the fastening element is a latching lug that engages in a locking manner under or in the measuring element when the first fastener is inserted into the insertion slot of the insertion channel in the cavity that is wider than the insertion slot. Other specific embodiments are self-evidently also conceivable, such as those in which the widened cavity is configured as a groove along the measuring element. In this context, the first fastener may generally be an insertable connector that may be inserted into the insertion channel such that the fastening element and the measuring element are in contact on at least one surface in the insertion direction. This contact results in transmission of the tensile forces and deformation of the measuring element, which is translated into movement data by the strain sensor.

The measuring element and thus also the strain sensor itself may be extended in the longitudinal direction of the insertion channel (e.g., due to the freely selectable insertion position) and, for example, cover the entire length of the insertion channel. It may therefore be provided that the measuring element and/or the sensor element covers the entire length of at least one insertion channel of the plurality of insertion channels. This may be provided if, in a development, the strain sensor is configured for spatially resolved measurement of the effect of the fastening element on the measuring element. It is then possible to determine which action (e.g., deformation) was caused by which first fastener, and, for example, also exactly where this first fastener is located (e.g., in the case of a plurality of first fasteners inserted over the length of the insertion channel).

However, in one embodiment, at least one of the insertion channels may have a plurality of measuring elements and/or sensor elements for different channel sections covering the entire length of the insertion channel. The channel sections may define insertion regions for individual securing straps. In this embodiment, the insertion channel is basically segmented, where each motion-sensitive channel section therefore has its own strain sensor. The sensor data of the strain sensors of all the insertion channels may be forwarded to the control device, and the evaluation with respect to the motion information may take place according to the position or rather the channel section of the strain sensor. For example, only some of the insertion channels (e.g., two outer insertion channels provided on the right and left, such as covering at least essentially the entire length of the patient table) may be segmented, while additional insertion channels (e.g., in inner areas of the patient table) may also have only a single measuring element or sensor element covering the entire length of these insertion channels. In this way, location information for sensor data may be obtained even if no spatially resolved measurement is possible with the strain sensors.

As already mentioned, in this context, the strain sensor may be configured for spatially resolved measurement of the effect of the fastening element on the measuring element. For example, the strain sensor may be configured as a fiber optic sensor with a fiber Bragg grating. A fiber Bragg grating (FBG) is a type of distributed Bragg reflector in which a periodic variation of the refractive index of the fiber core of the optical fiber is provided.

Since the fiber Bragg grating reacts sensitively to strain or general deformation, a fiber optic sensor may measure a shift in the Bragg wavelength. In order to allow a spatially resolved measurement, distributed fiber Bragg gratings are provided in the optical fiber, where a mechanical disturbance of the spacing of the fiber Bragg gratings shifts the local refractive index. This may be detected by an optical readout system at the end of the optical fiber. Such an arrangement has the advantage that even with a long optical fiber, which may be as long as the entire patient table or the longest insertion channel, for example, the exact position of the mechanical disturbance acting on the fiber core may be determined from the measured optical signals. Other types of fiber optic sensors may also be provided.

In general, various types of sensors may be used as strain sensors in the context of the present embodiments, as already explained in the introduction.

For example, it may be provided that at least one strain sensor of the plurality of strain sensors is configured as a rheostatic and/or capacitive strain sensor. In this case, bending or stretching of the measuring element is determined by strain gages that may, for example, be mounted in two or more sections of the measuring element. For example, a Wheatstone bridge may be created, as is generally known in the prior art. Capacitive strain sensors that are also known in the prior art may also be used. In general, rheostatic and/or capacitive sensors used as the rheostatic and/or capacitive strain sensor may have a tape and/or a flat braid and/or a cord as the sensor element.

In one embodiment, at least one strain sensor of the plurality of strain sensors includes at least one piezoelectric material as a sensor element. For example, a piezoelectric strain sensor of this kind, which is also well known in principle, may have distributed piezoelectric transducers that, for example, may also allow spatially resolved measurement.

With particular advantage, however, at least one (e.g., each) of the strain sensors may also generally have a sensor element having optical fibers as a measurement signal carrier (e.g., a sensor element based on stretchable optical fibers and/or a fiber Bragg grating sensor element). The advantage of optical fibers as measurement signal carriers in magnetic resonance imaging is that optical fibers are highly compatible with the electromagnetic radio frequency fields used there. This provides that interference with the imaging process may be prevented.

For example, deformation of the sensor element may be converted into an electrical signal by stretchable optical fibers. Similarly to conventional optical fibers, the stretchable optical fibers (e.g., elastic optical fibers) include a high refractive index core (e.g., made of a polystyrene-polyisoprene triblock copolymer) and a low refractive index cladding (e.g., made of a fluorinated thermoplastic elastomer). For example, the stretchable optical fibers may be repeatedly stretched up to three times their length. Axial deformation of the stretchable optical fibers results in a variation of their light guiding properties, which may be read by a light source and photodetector at the fiber ends.

Fiber optic sensors that use a fiber Bragg grating have already been described above, which self-evidently also applies generally.

In a development of the present embodiments, it may be provided that the control device is configured to compensate for movements (e.g., vibrations) caused by the operation of components of the magnetic resonance device (e.g., gradient coils) based on compensation information obtained in a calibration measurement. In the field of magnetic resonance imaging, for example, interfering vibrations (e.g., from gradient coils) may often occur. In order to improve the sensor data in this respect, it is possible to measure the vibrations caused by gradient activity (e.g., gradient pulses emitted via the gradient coils) in a calibration measurement (e.g., without a patient present, particularly deformations of the sensor element) and to use this compensation information during operation with a patient present to correct the sensor data (e.g., signals from the sensor element) accordingly. For example, the known magnetic resonance sequence running may be used to determine, from the compensation information, which interference signals or interference data are occurring, and these may be removed from the signals of the sensor element and/or sensor data by subtraction, for example. In this way, the quality of the sensor data is improved, and more robust evaluation is made possible.

In general, it may be provided for the control device to be configured to employ a trained evaluation function that uses the sensor data as input data to determine the motion information. In other words, artificial intelligence and/or machine learning (e.g., deep learning) may be used to enable fast and robust determination of the motion information.

A trained function maps generally cognitive functions that humans associate with other human brains. Through training based on training data (e.g., machine learning), the trained function is capable of adapting to new circumstances and detecting and extrapolating patterns.

Generally speaking, parameters of a trained function may be adapted through training. For example, supervised learning, semi-supervised learning, unsupervised learning, reinforcement learning, and/or active learning may be used. In addition, representation learning (e.g., also known as “feature learning”) may also be used. For example, the parameters of the trained function may be iteratively adapted by a plurality of training steps.

For example, a trained function may include a neural network, a support vector machine (SVM), a decision tree, and/or a Bayesian network, and/or the trained function may be based on k-means clustering, Q-learning, genetic algorithms, and/or assignment rules. For example, a neural network may be a deep neural network, a convolutional neural network (CNN), or a deep CNN. The neural network may also be an adversarial network, a deep adversarial network, and/or a generative adversarial network (GAN).

In the context of the present embodiments, provision may therefore be made to train an evaluation function (e.g., based on a large amount of experimental data) to detect and differentiate between various types of motion. For example, specific waveforms of respiration and/or heartbeat may be differentiated, where reinforcement learning may be used. In this context, provision may be made to acquire EKG signals and/or respiration signals together with sensor data as training input data of a training dataset and to provide them as ground truth or otherwise incorporate them into the learning process.

In a further embodiment, the measurement arrangement may additionally have at least one camera (e.g., a three-dimensional camera) capturing the patient on the patient table and/or at least one radar sensor detecting the patient on the patient table. The control device is configured to determine the motion information, additionally using camera data from the camera and/or radar data from the radar sensor.

In this embodiment, different ways of determining motion information are therefore combined, for example, to allow a statistical summary and/or mutual plausibility check. Image data from cameras, which may be provided anyway, and/or radar data of the patient may also be evaluated in order to improve the detection, localization, and quantification of patient motion. Three-dimensional cameras (e.g., TOF cameras, stereo cameras and/or terahertz cameras) are used with particular advantage to scan the patient in three dimensions and detect movements.

In principle, it is self-evidently also possible to use other sources of information and/or other specific motion information, such as navigator measurements from the magnetic resonance device, EKG measurements, motion information from respiratory belts, and the like, in order to improve the quality of the motion information.

Other sensors such as cameras and/or other sources of information may also be used to determine at least some of the background information mentioned in the introduction.

In addition to the measuring arrangement, the present embodiments also relate to a magnetic resonance device having a measuring arrangement according to the present embodiments. As already mentioned, the method presented here for determining patient motion information may be used particularly advantageously in magnetic resonance imaging because of the lengthy measurement times involved and the difficult electromagnetic environment. All the statement with regard to the measuring arrangement of the present embodiments also apply accordingly to the magnetic resonance device according to the present embodiments, so that the advantages already mentioned may also be achieved therewith. With particular advantage, the at least one strain sensor may be implemented in the patient table (e.g., as part of an insertion channel), so that the aforementioned motion detection may be achieved without the need to change other equipment (e.g., local coil arrays).

Also conceivable within the scope of the present embodiments (e.g., as a retrofit item) is a patient table that has a second fastening means (e.g., second fastener) for receiving a first fastening means (e.g., first fastener) of a securing strap. The second fastener has a strain sensor for measuring tensile forces of the securing strap that are introduced into the second fastener via the first fastener. With such a patient table, existing magnetic resonance devices or other medical treatment and/or examination equipment may be upgraded particularly easily and without the need to replace auxiliary devices, such as local coil arrays, in order to determine the motion information, provided that a corresponding update of the control device of the magnetic resonance device or other medical device is also carried out to evaluate the sensor data of the strain sensor with respect to the motion information.

The present embodiments also relate to a method for operating a measuring arrangement according to the present embodiments, where the motion information describing the movements of the patient is determined from the sensor data of the strain sensor using the control device. The method is thus ultimately used to measure the movements of a patient positioned recumbently on a patient table. All statements regarding the measuring arrangement according to the present embodiments and the magnetic resonance device according to the present embodiments are similarly applicable to the method according to the present embodiments, so that the advantages mentioned may also be achieved with this method. Since the method is carried out by the control device, the method is, for example, computer-implemented.

Accordingly, a computer program may also be provided within the scope of the present embodiments. The computer program, when executed on a control device of a measuring arrangement, causes the control device to carry out the acts of a method according to the present embodiments. The computer program may be stored on an electronically readable data carrier (e.g., a non-transitory computer-readable storage medium).

### BRIEF DESCRIPTION OF THE DRAWINGS

Further advantages and details will emerge from the example embodiments described below and from the accompanying drawings in which:

FIG. 1 shows a view of a patient positioned on a patient table in a first example embodiment of a measuring arrangement;

FIG. 2 shows a possible design of a securing strap;

FIG. 3 shows a magnetic resonance device with the measuring arrangement according to an embodiment;

FIG. 4 schematically illustrates an insertion channel with a securing strap;

FIG. 5 shows a sectional, schematic side view of the insertion channel of FIG. 4;

FIG. 6 shows a top view of the patient table of the magnetic resonance device; and

FIG. 7 shows a schematic view of a local coil array in a second example embodiment of a measuring arrangement.

### DETAILED DESCRIPTION

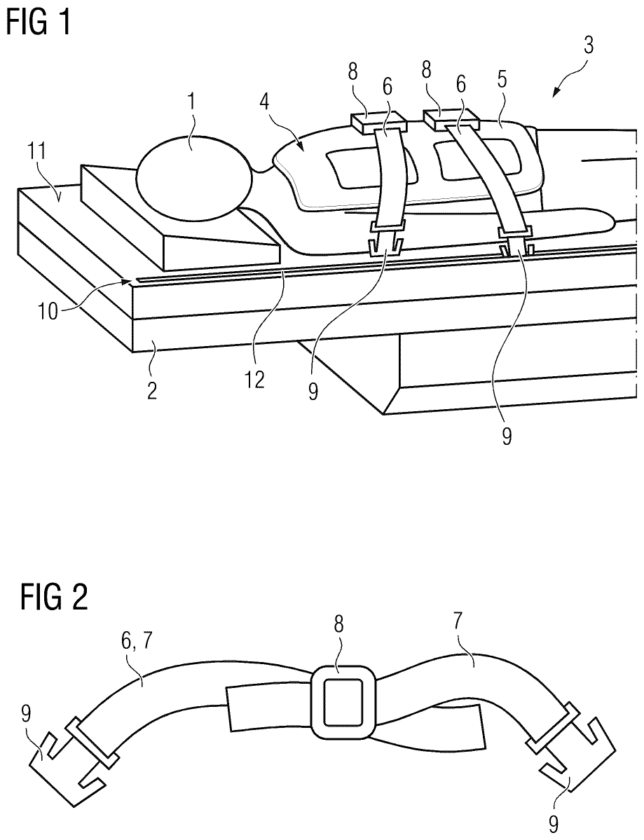

FIG. 1 shows a schematic view of a patient 1 positioned on a patient table 2 of a first example embodiment of a measurement arrangement 3. FIG. 1 shows that a local coil array 5 is provided as an auxiliary device 4 on the chest of the recumbently positioned patient 1. The local coil array is to be held in position by securing straps 6 during a magnetic resonance measurement.

In this first example embodiment, such a securing strap 6 is not part of the local coil array 5, but is provided as a separate component, as shown in more detail in FIG. 2. Accordingly, the securing strap 6 consists of, for example, two separate sections 7 that are connected via an adjustment device 8 (e.g., a buckle arrangement connecting the belt sections 7 in a displaceable and lockable manner) for adjusting the belt length available for securing. Other configurations may also be provided for the securing strap 6, such as using a single strap piece that may be adjusted in its strap length available for securing by the adjusting device 8.

At both free ends facing away from the adjustment device 8, the securing strap 6 has first fastener(s) 9 (e.g., insertable connectors). Returning to FIG. 1, these insertable connectors (e.g., the first fasteners 9) may engage with second fastener(s) 10 in the patient table that are configured as insertion channels 12 provided in the tabletop 11, to fasten the securing strap 6 to the patient table 2. Since such second fasteners 10 are provided on both sides of the patient 1 in the transverse direction (e.g., on the left side and right of the patient table 2), the securing strap 6 may be fastened on both sides with its corresponding first fastener(s) 9 such that the resulting tension is sufficient to hold the local coil array 5 in its current position and orientation during the magnetic resonance examination and not cause any discomfort to the patient 1.

The securing straps 6 are not extensible here (e.g., the securing straps 6 are at least essentially inelastic). This provides that tensile forces introduced into the securing straps 6 by movements of the patient 1 directly or indirectly via the local coil array 5 are transmitted via the securing straps 6 to the rigid first fasteners 9 where the tensile forces are transferred to the second fastener(s) 10 (not yet shown in FIG. 1 for the sake of clarity). Each of the securing straps 6 includes at least one strain sensor that, based on deformations of measuring elements that may correspond to the sensor elements of the respective strain sensor or are provided therewith, acquires sensor data through the tensile forces. The data may be evaluated to provide motion information describing the movement of the patient 1, as will be explained in more detail below.

Example embodiments in which different securing straps 6 are attached to at least one part of the auxiliary device 4 via a fastening device (e.g., to both sides) in order to thus allow the auxiliary device 4 to be secured in the respective position (and therefore also allowing the patient 1 to be secured locally) are provided. The advantage of separate securing straps 6 with first fasteners 9 on both sides is that the belt may also be directly placed taut over the patient 1 even without the auxiliary device 4 in order to transfer motion-induced tensile forces to the second fasteners 10 of the patient table 2 even without an auxiliary device 4 being present. For example, during a magnetic resonance measurement on the head of the patient 1, respiration may be measured by a securing strap 6 in the chest area; in addition, even with rigid, case-like local coil arrays, such as head coils and knee coils, motion measurement may be achieved (e.g., by having one of the securing straps 6) adjacent to the rigid local coil arrays that cannot be molded to the patient's shape, run directly over the patient 1 with a degree of tension that is not uncomfortable for the patient 1.

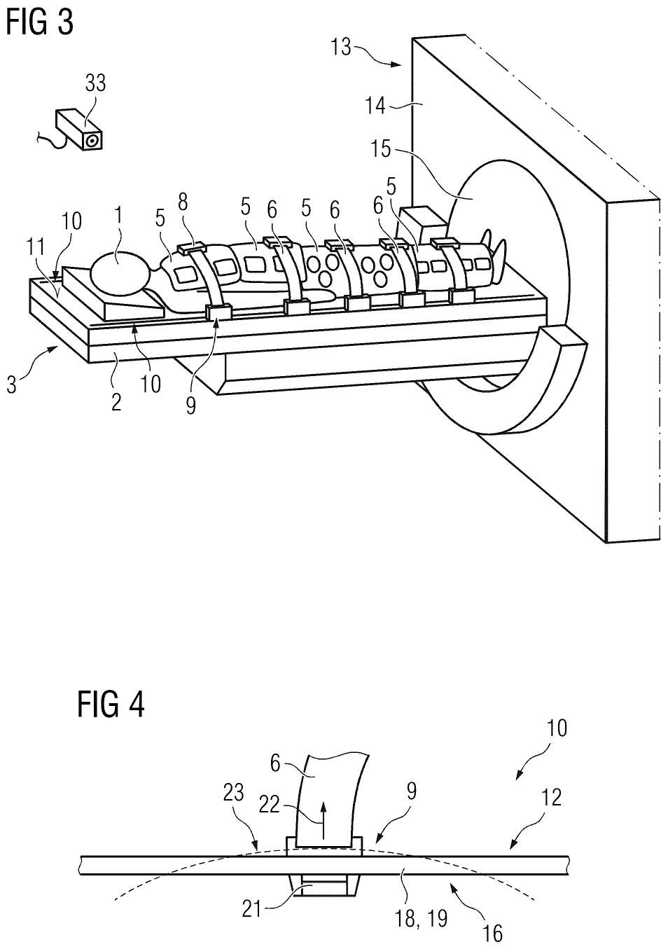

FIG. 3 shows an example of the possibility of using auxiliary devices 4 (e.g., local coil arrays 5) along the patient 1 for a whole-body examination with a magnetic resonance device 13 incorporating the measuring arrangement 3. The magnetic resonance device 13 includes a main magnet unit 14 with a patient bore 15 into which the patient table 2 may be moved. Using the respective securing straps 6 distributed over the length of the patient, motion measurements may be performed at various points (e.g., even where no imaging is currently taking place, such as outside the homogeneity volume).

FIGS. 4 and 5 schematically illustrate in more detail how the strain sensor 16 is incorporated into the second fasteners 10 in this case. FIG. 4 shows a schematic side view of the first fasteners 9 connected to the second fasteners 10, while FIG. 5 shows a schematic cross-section of the situation in FIG. 4.

The insertion channel 12, for example, has an insertion slot 17 that widens out below a measuring element 18 that, in the present case, also acts as a sensor element 19 and is correspondingly deformable to a certain extent, to form a cavity into which a fastening element 20 (e.g., a latching lug 21) of the first fastening fasteners 9 engages in a fastening manner such that at least the upper side of the latching lug 21 rests against the lower side of the measuring element 18 and thus, for example, prevents the first fasteners 9 from being pulled out of the insertion slot 17. In order to be able to release the connection again, an appropriate release may be provided for retracting the latching lug 21 or the like, as is known in the prior art in a variety of forms.

As already explained, the securing straps 6 are fastened such that a particular tension exists, so that the fastening element 20 is in firm contact with the measuring element 18. If movement of the patient 1 now occurs, a tensile force is generated, as indicated by the arrow 22 in FIG. 4. Due to the at least essentially non-extensible design of the securing strap 6 and the rigid design of the first fastening fasteners 9, the tensile force is transmitted to the latching lug 21 or more generally to the fastening element 20. The pressure with which the fastening element 20 bears against the measuring element 18 changes in response to the tensile force, resulting in the deformation of the measuring element 18 and, for example, simultaneously of the sensor element 19, which is measured by the strain sensor 16. This deformation is indicated by the dashed line 23 in FIG. 4 for illustrative purposes.

In the present example embodiment, the strain sensor 16 used is an optical fiber sensor that therefore uses at least one optical fiber 24, also indicated in FIG. 5, in the sensor element 19. In the present case, the sensor element 19 uses distributed fiber Bragg gratings (FBG) as already described above, so that a spatially resolved measurement is possible (e.g., it is possible to determine in the sensor data where the tensile force or change in tensile force described by the arrow 22 is acting). This may be provided if a plurality of first fasteners 9 are inserted along the section of the insertion channel 12 in which the measuring element 18 extends, which is possible due to the free choice of insertion location.

When using an optical fiber sensor that provides spatially resolved sensor data, the entire length of the insertion channels 12 (e.g., including the outer ones) may be covered with a single measuring element 18. However, as shown by way of example in FIG. 4 and FIG. 5, a plurality of measuring elements 18 may be provided and therefore strain sensors 16 along at least one of the insertion channels 12 of the patient table 2, each covering a channel section.

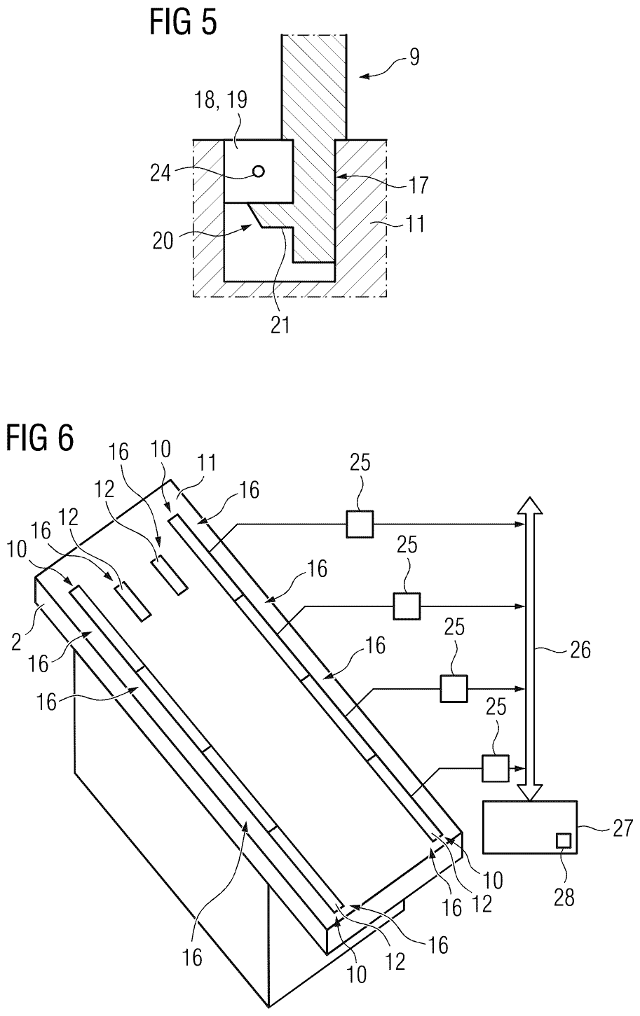

In this context, FIG. 6 schematically shows a perspective view of the patient table 2. FIG. 6 shows that insertion channels 12 extending at least essentially over the entire useful length of the patient table 2 are provided at outer right and left as second fasteners 10. In the region in which the head of the patient 1 is to be placed, two further insertion channels 12 are shown. Corresponding further, shorter insertion channels 12 may also be provided in the region of the arms/hands and/or in the region of the legs/feet, but are not shown for the sake of clarity. While only a single measuring element 18 is provided for the shorter insertion channels 12 in each case for the entire length of the insertion channel 12, and thus a single strain sensor 16, the outer insertion channels 12 are divided into four channel sections, in each of which a measuring element 18 is present, so that each of these channel sections includes its own strain sensor 16.

In one embodiment, measurements may be made on both sides of the patient 1 in each case (e.g., right and left) in order to obtain a coherent overall picture from the sensor data that, in addition to a more precise determination of the movements of the patient, may also allow a plausibility check.

The sensor data of the strain sensors 16 are determined in control units 25 including corresponding electronic components that, for the sake of clarity, are only shown in FIG. 6 for the strain sensors 16 of the outer right insertion channel 12 (e.g., from corresponding optical signals in the sensor element 19, such as the optical fiber 24, which signals may be generated by a laser and may be measured by a photodetector). The control units 25 may also be installed in the patient table 2 and transmit their sensor data, such that the transmitting strain sensor 16 may be identified, via a bus 26 to a control device 27 that determines the motion information from the sensor data (e.g., analyzing them in combination and taking their origin into account). In the present case, the control device 27 is the control device of the magnetic resonance device 13.

The control device 27 has a memory device 28 and at least one processor (not shown in detail). For evaluation purposes, the control device 27 may include at least one evaluation unit whereby at least one evaluation function is applied to the sensor data as input data in order to obtain the motion information as output data.

However, before the sensor data is evaluated, the sensor data is corrected for interference from components of the magnetic resonance device (e.g., the gradient coils) based on compensation information present in the memory device 28. A correction unit may be provided for this purpose. The gradient coils of the gradient coil array of the magnetic resonance device 13 may introduce vibrations into the patient table 2, which are also measured. Consequently, in a calibration measurement, compensation information without a patient 1 was acquired by the strain sensors 16 with the gradient coils being operated using particular operating parameters, so that the correction unit may remove corresponding interference contributions in the sensor data (e.g., by subtraction).

As part of the evaluation process, the motion information may be determined such that the motion information describes the strength and location of the movement of the patient 1 for at least one type of motion. For example, the at least one type of movement may include a cyclical type of motion (e.g., respiratory motion and/or movements due to heartbeat) and/or an unconscious type of movement (e.g., muscle spasms) and/or a conscious type of movement.

With particular preference, a trained evaluation function is used to detect and separate different motion types and enable their strength and/or location to be determined. The trained evaluation function may be trained in a training phase using training datasets in which sensor data from the strain sensors 16 (e.g., as training input data) may be assigned to ground truths (e.g., as training output data) that have been obtained by other measuring method(s) and/or device(s), for example. Reinforcement learning may be used, for example, in connection with EKG and respiration measurements when considering cyclical movement types.

The control device 27, which also serves as the control device of the magnetic resonance device 13, may use the motion information in a variety of specific ways (e.g., to trigger an acquisition process for periods of low motion, to mark magnetic resonance data contaminated by motion, and/or to correct acquired magnetic resonance data for motion).

Variations of the specific example embodiment of the measuring arrangement 3 described here are also possible. For example, instead of the (extended) cavity 19, a groove that accommodates the fastening element 20 may also be provided in the measuring element 18, so that, for example, forces may be transmitted and measured in both directions.

If the measuring element 18 does not itself constitute the sensor element 19, the sensor element 19 may also be applied to the measuring element 18 using a soft, extensible (e.g., tape-like) material.

In addition, other types of strain sensors 16 may also be used, such as conventional rheostatic and/or capacitive strain sensors and/or strain sensors using piezoelectric materials.

In one embodiment, however, fiber optic strain sensors 16 are used in the context of magnetic resonance, where, in addition to the described embodiment with distributed fiber Bragg gratings, those with stretchable optical fibers may also be used.

It is also possible for strain sensors 16 to be accommodated in other ways (e.g., with the sensor element 19 on the securing strap 6).

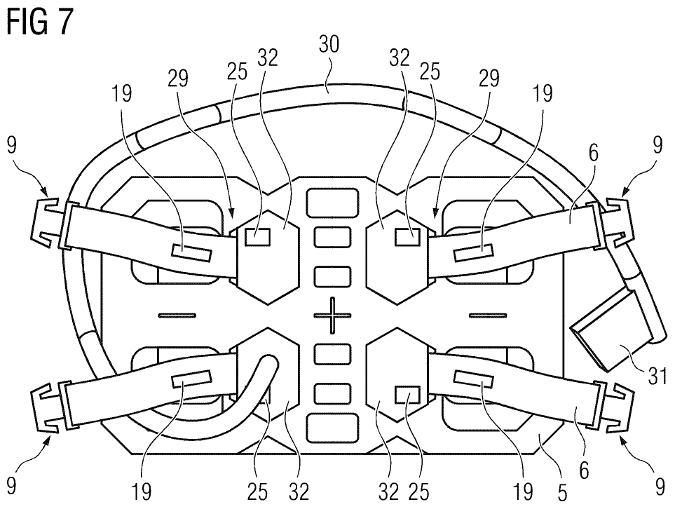

In this context, FIG. 7 illustrates, as an essential component of a second example embodiment of a measuring arrangement 3, a local coil array 5 to which four securing straps 6 are fastened, two on each side, using fastening devices 29 with first fasteners 9 provided at the other ends thereof. In addition, the local coil array 5 also has an electrical connecting cable 30 with a corresponding coil connector 31.

The sensor elements 19 of the strain sensors 16 are attached to or incorporated in an extensible portion of the securing straps 6. The control units 25 of the strain sensors 16 are implemented as part of electronic units 32 of the local coil array 5, on which units the fastening devices 29 are also provided to simplify the establishment of the wired communication connection between the sensor element 19 and the control unit 25.

In addition to a wired transmission path between the sensor element 19 and the control unit 25, wireless communication options may also be used in other example embodiments in which the sensor element 19 is provided on the securing strap 6. In any case, it is evident that the sensor element 19 may be disposed adjacent to the fastening device 29.

Further, a similar design may also be realized with respect to the patient table 2 with the sensor element 19 disposed adjacent to the first fastening means 9. For example, the control unit 25 may be provided on or adjacent to the second fasteners.

Additional measuring device(s) or sources of information may also be taken into account by the control unit 27 in the same way as existing background information. For example, motion information may be determined in other ways and combined statistically or plausibly with the motion information of the strain sensors 16.

For example, in both example embodiments, the measuring arrangement 1 may also include at least one camera 33, indicated in FIG. 3, which may measure in three dimensions and from which image data its own motion information may be determined, since the at least one camera 33 captures the patient 1. However, it is also possible for the image data of the camera 33 to be used to determine background information for determining the motion information from the sensor data of the strain sensors 16 (e.g., where which securing strap 6 is located, how it runs, and the like).

Although the invention has been illustrated and described in detail by the example embodiments, the invention is not limited by the disclosed examples, and other variations will be apparent to persons skilled in the art without departing from the scope of protection sought for the invention.

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A measuring arrangement for measuring movements of a patient positioned recumbently on a patient table, the measuring arrangement comprising:
the patient table;
at least one flexible securing strap for locally securing the patient on the patient table, wherein the at least one flexible securing strap comprises at least one first fastener that co-operates with a second fastener of the patient table for fastening the at least one flexible securing strap;
a strain sensor provided as part of the at least one flexible securing strap, motion-coupled to the at least one flexible securing strap, or a combination thereof; and
a control device configured to determine motion information describing the movements of the patient from sensor data of the strain sensor.

2. The measuring arrangement of claim 1, further comprising an auxiliary device that is disposable on the patient and securable to the patient,
wherein the at least one flexible securing strap comprises a plurality of flexible securing straps,
wherein:
at least two securing straps of the plurality of flexible securing straps are fastened to the auxiliary device by an end opposite the at least one first fastener by a fastening device;
at least one securing strap of the plurality of flexible securing straps has first fasteners on both sides for oppositely disposed second fasteners of the patient table; or
a combination thereof.

3. The measuring arrangement of claim 1, wherein a sensor element of the strain sensor is provided on an elastic portion of the at least one flexible securing strap.

4. The measuring arrangement of claim 3, wherein the sensor element is a strain gage.

5. The measuring arrangement of claim 1, wherein the at least one flexible securing strap has an adjuster that is configured for adjusting a belt length of the at least one flexible securing strap, is inelastic, or a combination thereof.

6. The measuring arrangement of claim 1, further comprising a plurality of strain sensors, the plurality of strain sensors comprising the strain sensor,
wherein at least one strain sensor of the plurality of strain sensors is provided by a first fastener of the at least one first fastener, the second fastener, or the first fastener and the second fastener.

7. The measuring arrangement of claim 6, wherein the first fastener has a fastening element that, in the fastened state, is in contact with a measuring element of the second fastener, and
wherein the measuring element is configured as a sensor element of the strain sensor, or the sensor element of the strain sensor is disposed on the measuring element.

8. The measuring arrangement of claim 7, wherein the fastening element is a latching element.

9. The measuring arrangement of claim 7, wherein the second fastener is configured as an insertion channel for inserting the first fastener in any position covered by the insertion channel.

10. The measuring arrangement of claim 9, wherein the position covered by the insertion channel is a longitudinal direction of the patient table.

11. The measuring arrangement of claim 9, wherein the measuring element forms a limit of an insertion slot of the insertion channel and of a cavity configured to receive the fastening element and widened with respect to the insertion slot.

12. The measuring arrangement of claim 9, wherein the measuring element, the sensor element, or the measuring element and the sensor element cover an entire length of at least one insertion channel of a plurality of insertion channels, or at least one insertion channel of the plurality of insertion channels has a plurality of measuring elements, sensor elements, or measuring elements and sensor elements for different channel sections covering the entire length of the insertion channel.

13. The measuring arrangement of claim 12, wherein the strain sensor is configured for spatially resolved measurement of an effect of the fastening element on the measuring element.

14. The measuring arrangement of claim 13, wherein the strain sensor is configured as a fiber-optic sensor with a fiber Bragg grating.

15. The measuring arrangement of claim 1, wherein the strain sensor has a sensor element having optical fibers as a measurement signal carrier.

16. The measuring arrangement of claim 15, wherein the sensor element is based on stretchable optical fibers, is a fiber Bragg grating sensor element, or is a combination thereof.

17. The measuring arrangement of claim 1, wherein the control device is further configured to compensate for movements in the sensor data caused by operation of components of a magnetic resonance device, based on compensation information obtained in a calibration measurement.

18. The measuring arrangement of claim 1, wherein the control device being configured to determine the motion information comprises the control device being configured to employ a trained evaluation function using the sensor data as input data.

19. The measuring arrangement of claim 1, further comprising:
at least one three-dimensional (3D) camera configured to capture the patient on the patient table;
at least one radar sensor configured to detect the patient on the patient table; or
a combination thereof,
wherein the control device is further designed to determine the motion information additionally using camera data from the at least one camera, radar data from the at least one radar sensor, or the camera data and the radar data.

20. A magnetic resonance device comprising:
a measuring arrangement for measuring movements of a patient positioned recumbently on a patient table, the measuring arrangement comprising:
the patient table; and
at least one flexible securing strap for locally securing the patient on the patient table, wherein the at least one flexible securing strap comprises at least one first fastener that co-operates with a second fastener of the patient table for fastening the at least one flexible securing strap;
a strain sensor provided as part of the at least one flexible securing strap, motion-coupled to the at least one flexible securing strap, or a combination thereof; and
a control device configured to determine motion information describing the movements of the patient from sensor data of the strain sensor.

21. A method for operating a measuring arrangement, the measuring arrangement being for measuring movements of a patient positioned recumbently on a patient table, the measuring arrangement comprising the patient table, at least one flexible securing strap for locally securing the patient on the patient table, wherein the at least one flexible securing strap comprises at least one first fastener that co-operates with a second fastener of the patient table for fastening the at least one flexible securing strap, a strain sensor provided as part of the at least one flexible securing strap, motion-coupled to the at least one flexible securing strap, or a combination thereof, and a control device configured to determine motion information describing the movements of the patient from sensor data of the strain sensor, the method comprising:
determining the motion information describing the movements of the patient from the sensor data of the strain sensor using the control device.
